(19) United States
(12) Patent Application Publication
(10) Pub. No.: US 2015/0133547 A1
(43) Pub. Date: May 14, 2015

(54) METHOD AND COMPOSITION FOR TREATING GLAUCOMA

(71) Applicant: Richard W. Hertle, Hudson, OH (US)

(72) Inventor: Richard W. Hertle, Hudson, OH (US)

(21) Appl. No.: 14/075,223

(22) Filed: Nov. 8, 2013

(51) Int. Cl. C07C 57/62

(52) U.S. Cl. CPC C07C 57/62
USPC 514/531; 560/124

(57) ABSTRACT

Methods for reducing intra-ocular pressure and for treating eye disorders characterized by increased intra-ocular pressure, such as glaucoma, by ophthalmic administration of a pharmaceutical composition comprising a pyrethroid compound that is lambda-cyhalothrin or an analog thereof through topical administration of the composition or incorporation into an intraocular device. Pharmaceutical compositions for the treatment of such eye disorders comprising lambda-cyhalothrin or an analog thereof and methods for making them are disclosed.

### FIELD OF THE INVENTION

The present invention is directed to a method for reducing intraocular pressure (IOP) and treating glaucoma. The method involves the use of particular pyrethroid compounds. The present invention is also directed to pyrethroid-based compositions for use in the method.

### BACKGROUND OF THE INVENTION

Glaucomas are a group of ophthalmic disorders or conditions characterized by elevated intraocular pressure (IOP). Glaucomas may be classified as open angle or angle-closure glaucoma based upon the anatomy of the eye's anterior chamber. Glaucomas may be further classified as primary or secondary, depending on the cause.

The eye is filled with aqueous humor, which helps to maintain the global shape of the eye, supplies nutrition to ophthalmic structures, such as the lens and cornea, and transports waste material away from the eye. Aqueous humor is a flowing liquid. IOP is the pressure generated by the flow of aqueous humor against resistance within ocular structures. Normally, IOP is maintained at around 15 mmHg in healthy individuals. IOP reflects a balance between the rates that aqueous humor enters and leaves the eye. Inflow of aqueous humor is dependent upon the rate of production of aqueous humor, whereas outflow is determined by resistance to drainage from the eye.

Elevated IOP results in damage to retinal ganglion cells that further result in a typical pattern of loss in the field of vision. Vision loss may progress to tunnel vision and blindness, if the elevated IOP is left untreated. Not surprisingly, current treatments are all directed toward reducing IOP. Raised intraocular pressure (above ˜21 mmHg or 2.8 kPa) is the most important and only modifiable risk factor for glaucoma.

Currently available treatments include surgical and pharmacological treatments. Surgical procedures include laser trabeculoplasty to increase outflow, tube shunts and a trabeculotomy to create additional outflow routes, and destruction of ciliary body epithelial tissues to reduce aqueous humor production. Surgical procedures for treating glaucoma disorders are beyond the field of this invention. The disadvantages of surgery included complications associated with intraocular procedures and typically include: hemorrhage, infection, cataract, retinal detachment, loss of vision and reactions to anesthesia.

Pharmacological therapies are effective for reduction of IOP and typically include two types of agents, those that decrease production of aqueous humor and those that increase the outflow or removal of aqueous humor from the eye. Either or both mechanisms can result in reduced IOP. A wide variety of agents are available, most of them in ophthalmic solution form. Other dosage forms include suspensions, gels, ointments and drug-containing ocular implants.

Some common examples of drugs used to treat elevated IOP and/or glaucoma include parasympathomimetic agents, sympathomimetic agents, prostaglandin analogs, beta blockers, alpha agonists, cholinesterase inhibitors, and carbonic anhydrase inhibitors. Often more than one type of drug is utilized.

Useful parasympathomimetic agents include pilocarpine and carbachol; useful cholinesterase inhibitors include echothiophate iodide (also known as phospholine iodide). These types of agents reduce IOP by increasing aqueous humor outflow through the trabecular meshwork of the eye. Sympathomimetic agents, such as epinephrine (no longer available in the United States) and dipivefrine, also increase outflow of aqueous humor from the eye.

Pilocarpine, carbachol, phospholine iodide and dipivefrine are no longer routinely used as their long-term use leads to many untoward complications (i.e., allergy, pain, miosis, cataracts, and retinal detachment) and are now replaced by newer, safer more effective agents.

Prostaglandin analogs also work by increasing outflow of the aqueous humor from the eye. Useful prostaglandin analogs include bimatoprost, latanoprost and travoprost, commercially available as Lumigan®, Xalatan® and Travatan®, respectively. While the prostaglandin analogs are generally regarded as safe and effective, with fewer systemic side effects than other agents, they do cause changes to the eye itself, including changes to the color of the iris and eyelash growth.

Beta blockers work by decreasing production of aqueous humor. Useful beta blockers include betaxolol, timolol, carteolol, metipranolol, and levobunolol, available commercially under a number of brand names. They are less expensive than the prostaglandin analogs, but have more systemic side effects. Side effects can be minimized by various techniques used for drop application that reduce the amount of drug entering the tear duct and systemic circulation.

Alpha agonists, such as apraclonidine and brimonidine, work to decrease production of aqueous humor and increase its drainage from the eye.

Carbonic anhydrase inhibitors, such as acetazolamide, brinzolamide, dorzolamide and methazolamide, decrease the production of aqueous humor. Brinzolamide and dorzolamide are available as eye drops, and acetazolamide and methazolamide are available in solid oral forms.

Drug therapies may be combined for optimal effect. And, to reduce the number of eye drops used per day and the number of prescription containers to keep track of, many combination formulations, containing more than one drug, are available to provide convenience to the patient. These combination formulations may also be more cost effective.

There are several new drugs undergoing development for use in treating the glaucomas, with particular emphasis on reducing IOP. Among these include gel forms of the more traditional agents, derivatives of clonidine, newer prostaglandins, alpha tocopherol, phenyloin, aminoguanidine and the N-methyl-d-aspartate receptor antagonist, memantine. There is continuing interest in developing cost-effective therapies that can lower IOP over a longer period of time, increase adherence by reducing dosing frequency and minimizing local side effects, and stop loss of vision. Research into such therapies is ongoing.

Traditional therapies for the glaucomas do not provide a cure, and they are not without some attendant disadvantages as discussed above. In addition, many traditional treatments, even combination formulations, require frequent doses per day, thus increasing the chances for patient non-compliance and the cost of the therapy. Local side effects are not uncommon and include local inflammation, burning, stinging, tearing, blurred vision, foreign body sensation, dry eyes, among other effects. Systemic effects include low heart rate and blood pressure, anticholinergic effects, headache, body aches, joint pain, flu-like syndromes, and the like, depending on the agent. Some agents, such as non-specific beta blockers, are contraindicated in asthma and chronic obstructive pulmonary disease. There is a continuing need, therefore, for new therapeutic agents that provide prolonged control of IOP with fewer local and systemic side effects than traditional therapies.

A novel treatment for the control of elevated IOP has been discovered that utilizes certain topical pyrethroids, not heretofore used as a pharmacological agent for the treatment of the glaucomas or to reduce IOP. The treatment method includes the use of compositions comprising pyrethroid chemicals, in particular lambda-cyhalothrin and analogs thereof.

Pyrethroids are a group of man-made chemicals similar to the natural insecticide pyrethrins produced by chrysanthemum flowers. Pyrethroids disrupt the normal functioning of the nervous system in an organism. In particular, pyrethroids disrupt the nervous system of insects by prolonging the deactivation of voltage-gated sodium channels, which results in prolonged excitation of nerve fibers. Through nervous system disruptions, pyrethroids may cause paralysis or death.

Synthetic pyrethroids generally have improved properties and effects over natural pyrethroids. Particular synthetic pyrethroids, for example those having alpha-cyano group (such as lambda-cyhalothrin, the active ingredient in Hot Shot® Home Insect control (Spectrum Brands) and Warrior IIP® crop protection pesticide (Syngenta)), have more potent neurotoxic effects than pyrethroids that do not contain an alpha-cyano group.

Lambda-cyhalothrin is an Environmental Protection Agency (EPA)-registered insecticide that is similar to the pyrethroid cyhalothrin and particularly useful as an insecticide. Lambda-cyhalothrin is a colorless-to-beige solid with a mild odor, having low water solubility and low volatility.

Like other pyrethroids, lambda-cyhalothrin repels insects and affects a large variety of insects that consume or otherwise come into contact with the chemical. A large number of products contain lambda-cyhalothrin, including but not limited to indoor and outdoor insecticides, such as agricultural insecticides for crops; insecticides for home, hospital, and other closed environments; greenhouse, ornamental plant and lawn insecticides; termite treatments; and animal treatments. Some commercially available products containing lambda-cyhalothrin include the Demand®, Karate®, and Warrior® brands.

Before pesticides are registered by the U.S. EPA, they must undergo testing for short-term and long-term health effects to determine how the chemical affects the body in cases of overexposure. The EPA classifies lambda-cyhalothrin as moderately toxic when inhaled and moderately toxic in skin effects for rats and for eye effects. Yet, lambda-cyhalothrin has low toxicity in skin effects for rabbits and no skin sensitivity in guinea pigs.

Humans working with lambda-cyhalothrin in laboratories reported symptoms of facial tingling and burning, beginning within 30 minutes of exposure and lasting 6 hours to 2 days. All incidents involved handling of pure or concentrated lambda-cyhalothrin. Overall, when used according to label directions, toxic effects are less likely to occur, because exposure amounts are much lower compared to doses used in laboratory toxicity testing. Reported effects by users of lambda-cyhalothrin-containing products include: irritation to the skin, throat, nose, and other body parts, if exposed. Skin tingling, burning, and prickly feelings, particularly around the face, are unique temporary symptoms of exposure. Other symptoms may include dizziness, headache, nausea, decreased appetite, and fatigue. In severe poisoning, seizures and coma may occur.

Notwithstanding the potential and known side effects of pyrethroid exposure, unexpectedly, the present inventive compositions, comprising effective doses of lambda-cyhalothrin and analogs thereof, provide unexpected and prolonged reduction of IOP with little to no observed local or systemic toxic effects in the doses utilized.

It is an object of the invention to provide a composition useful in the treatment of ocular conditions characterized by increased intraocular pressure, such as glaucoma.

It is a further object of the invention to provide a method for the treatment of glaucoma that provides for significant and prolonged reduction of IOP with little or no toxic local or systemic side effects.

Another object of the invention is to provide a composition comprising a pyrethroid compound in a suitable delivery system for administration into the eye by topical administration or implantation or insertion of an ocular device.

Still other objects of the invention will be evident from the disclosure herein.

### SUMMARY OF THE INVENTION

The present invention is directed to the use of a pyrethroid compound to reduce intra-ocular pressure and, more particularly, to treat glaucoma and ocular diseases associated with increased intraocular pressure. It is based on the discovery that exposure to lambda-cyhalothrin resulted in a significant reduction of intra-ocular pressure that persists for hours to days. The inventive compositions, in use, have few to no systemic side effects and a comparable or better local side effect profile.

In one embodiment, the present invention is a method to decrease intra-ocular pressure in a human comprising the step of administering an effective amount of a pyrethroid compound, contained in a suitable ophthalmic carrier or delivery system, into the eye. In particular, the pyrethroid compound is lambda-cyhalothrin or an analog thereof containing an alpha-cyano group.

In another embodiment, the present invention is a method of treating glaucoma and other conditions characterized by increased IOP in a human comprising the step of administering an effective amount of a pyrethroid compound, contained in a suitable ophthalmic carrier or delivery system, into the eye. In particular, the pyrethroid compound is lambda-cyhalothrin or an analog thereof.

In still another embodiment, the present invention is directed to a pharmaceutical composition comprising a pyrethroid compound in a liquid, semi-liquid or solid carrier, in an amount sufficient to reduce intra-ocular pressure to within normal limits.

In yet another embodiment, the present invention is directed to a method for preparing a pharmaceutical composition useful in the treatment of increased IOP and/or glaucoma

The present invention is described in terms of the use of lambda-cyhalothrin; however, it is not limited as such. Other suitable pyrethroids include: permethrin, cypermethrin, deltamethrin, bifenthrin, cyfluthrin, prallethrin, resmethrin, sumithrin, tetramethrin, and tralomethrin. These pyrethroids are available commercially in a large number of insecticide products. It is believed that pyrethroids containing alpha-cyano groups are particularly effective in the inventive method. While not wishing to be bound by any particular theory, it is believed that the mechanism of intraocular pressure reduction is localized to the non-pigmented ciliary epithelium of the ciliary body or uveo-scleral outflow system. The mechanisms are under investigation.

### DETAILED DESCRIPTION OF THE INVENTION

The present invention is directed to a method for reducing intra-ocular pressure to within normal limits and, in particular, for treating glaucoma, comprising the step of administering a topical or implantable ophthalmic preparation comprising a pyrethroid compound in an amount sufficient to reduce IOP to within normal range for a human subject. The invention is also directed to a composition comprising a pyrethroid, lambda-cyhalothrin or an analog thereof, provided as an ophthalmic preparation and a method for the preparation of the composition.

The present invention is described in terms of lambda-cyhalothrin. Lambda-cyhalothrin is represented by the formula, C₂₃H₁₉ClF₃NO₃, as set forth below in Formula I:

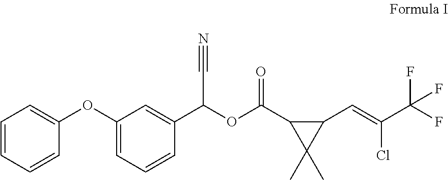 [SMILES: CC1(C)C(/C=C(\Cl)C(F)(F)F)C1C(=O)OC(C#N)c1cccc(Oc2ccccc2)c1]

Lambda-cyhalothrin is also known by its chemical name (R)-cyano(3-phenoxyphenyl) methyl (1S,3S)-rel-3-[(1Z)-2-chloro-3,3,3-trifluoro-1-propenyl]-2,2-dimethyl cyclopropane carboxylate.

Other useful compounds are lambda-cyhalothrin analogs having Formulas II-VII below:

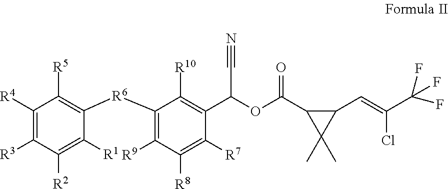

Where R¹-R⁵ and R⁷-R¹⁰ each may be the same or different (relative to any other of R¹-R⁵ and R⁷-R¹⁰) and may be hydrogen (H), alkyl (e.g. C₁-C₄ alkyl, methyl, ethyl), alkoxy (e.g. C₁-C₄ alkoxy, methoxy, ethoxy), hydroxyl, or halogen (e.g. F, Cl, Br, I), and any one of R¹-R⁵ and R⁷-R¹⁰ is preferably H; and

Where R⁶ may be oxygen (O), nitrogen (N), or alkyl (e.g. C₁-C₄ alkyl) and preferably is O.

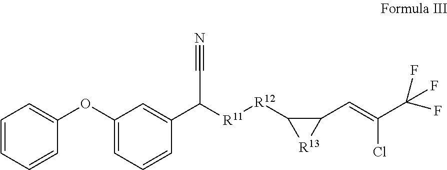

Where R¹¹ may be O or N or alkyl (e.g. C₁-C₄ alkyl) and preferably is O;

Where R¹² may be C═O (ketone) or C—OH or unbranched or branched alkyl (e.g. C₁-C₄ alkyl) or C—SO₂ or C—SH (sulfhydryl) and preferably is C═O; and

Where R¹³ is substituted or unsubstituted alkyl (e.g. C₁-C₄ alkyl) or substituted or unsubstituted CH₂—NH—CH₂, or a pyrrole ring; wherein one or more substituents, if present, may be alkyl (e.g. C₁-C₄ alkyl, methyl, or ethyl), alkoxy (e.g. C₁-C₄ alkoxy, methoxy, ethoxy), hydroxyl, or halogen (e.g. F, Cl, Br, I); and preferably is C(CH₃)₂.

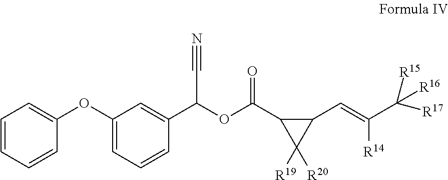

Where R¹⁴-R²⁰ each may be the same or different (relative to any other of R¹⁴-R²⁰) and may be hydrogen (H), alkyl (e.g. C₁-C₄ alkyl, methyl, ethyl), alkoxy (e.g. C₁-C₄ alkoxy, methoxy, ethoxy), hydroxyl, or halogen (e.g. F. Cl, Br, I), and R¹⁴ preferably is Cl, any one of R¹⁵-R¹⁷ preferably is F, and any one of R¹⁹ and R²⁰ preferably is alkyl (e.g. C₁-C₄ alkyl) and more preferably is methyl.

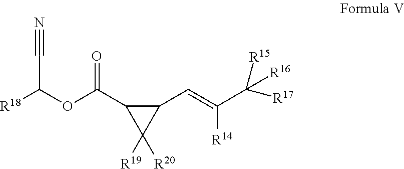

Where R¹⁸ may be a substituted or unsubstituted aryl, a substituted or unsubstituted naphthyl, a substituted or unsubstituted purine, a substituted or unsubstituted pyrimidine, a substituted or unsubstituted pyrrole, a substituted or unsubstituted biphenyl, a substituted or unsubstituted cyclic alkyl, a substituted or unsubstituted bicyclic alkyl, or a substituted or unsubstituted bicyclic compound where two aromatic rings, preferably two phenyl rings, are joined via an alkyl (e.g. C₁-C₄ alkyl, methyl, ethyl), O or N (preferably phenoxyphenyl); wherein one or more substituent, if present, may be alkyl (e.g. C₁-C₄ alkyl, methyl, or ethyl), alkoxy (e.g. C₁-C₄ alkoxy, methoxy, ethoxy), hydroxyl, or halogen (e.g. F, Cl, Br, I), and preferably is an unsubstituted phenoxyphenyl joined to the rest of the molecule at the 3 position; and

Where R¹⁴-R²⁰ are as set forth above.

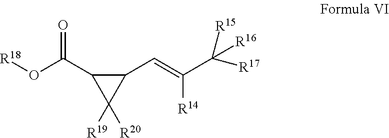

Where R¹⁸ and R¹⁴-R²⁰ are as set forth above.

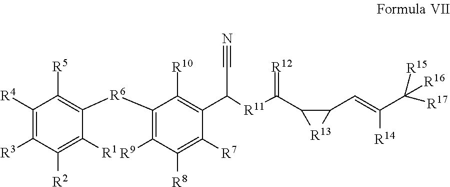

Where substituents R¹-R¹⁷ are as set forth above.

The inventive compositions include a pyrethroid, as described above in any of Formulas I-VII, in a suitable ophthalmic carrier or delivery system, present in an amount sufficient to reduce intraocular pressure to within normal ranges.

The compositions of the present invention comprise lambda-cyhalothrin or one of its analogs in amounts ranging from about 0.001 to about 5% w/w (weight of pyrethroid/weight of total composition), preferably between about 0.001 to about 1.0% w/w or more preferably between about 0.01 to about 1.0% w/w.

The inventive compositions may utilize solutions, suspensions, ointments, emulsions or gels as carriers or delivery systems for the pyrethroid compound. Useful liquid carriers include balanced salt and artificial tear solutions. Other useful liquid carriers or delivery systems for ophthalmic medications will be known to one skilled in the art. Alternatively, the pyrethroid component may be imbedded or incorporated in an ocular implant device, including but not limited to a reservoir, matrix, a contact lens or other solid device.

The pharmaceutical compositions of the invention may include various other ingredients, including but not limited to one or more surfactants, tonicity agents, buffers, antimicrobial agents, preservatives, co-solvents and/or viscosity building agents or thickeners. Suitable ingredients are known to one skilled in the art of ophthalmic pharmaceuticals.

Suitable surfactants include, but are not limited to, surface-active agents that are traditionally used in ophthalmic applications, such as polysorbate 80 (commercially available as TWEEN® 80 (ICI America Inc.)), tyloxapol, as well as PLURONIC® F-68 (BASF) and other poloxamer surfactants. These latter two surfactants are nonionic polyalkylene oxide block copolymers useful to improve water solubility and/or miscibility of hydrophobic compounds. The concentration in which any particular surfactant is used may be limited by its neutralization of the bactericidal effects of the preservatives (if present) or by levels that are cause irritation. Other useful surfactants and acceptable concentrations for ophthalmic preparations will be known to one skilled in the art.

Various tonicity agents useful to adjust the tonicity of the composition include: sodium chloride, potassium chloride, magnesium chloride, calcium chloride, nonionic diols, preferably glycerol, dextrose and/or mannitol may be added to the composition to approximate physiological tonicity. Other useful tonicity agents will be known to one skilled in the art. The amount of tonicity agent useful in the invention may vary, depending on the particular agent to be added. In preferred non-limiting embodiments of the invention, a pharmaceutical composition comprises a tonicity agent in an amount sufficient to cause the final formulation to have an ophthalmically acceptable osmolality (generally about 150-450 mOsm).

An appropriate buffer system, including but not limited to sodium phosphate, sodium acetate, sodium citrate, sodium borate, or boric acid, may also be used to reduce or prevent pH drift under storage conditions. Other appropriate buffer systems would be known to one skilled in the art. The particular concentration used would vary depending on the agent employed.

In some preparations, it may be desirable to include a preservative or antimicrobial agent to reduce, limit or prevent microbial contamination. Suitable preservatives include, but are not limited, to: biguanides, hydrogen peroxide, hydrogen peroxide sources, benzalkonium chloride, chlorobutanol, benzododecinium bromide, methylparaben, propylparaben, phenyl ethyl alcohol, edetate disodium, sorbic acid, polyquaternium-1, and/or other preservative or antimicrobial agents known to be useful to those skilled in the art. In specific non-limiting embodiments, a preservative is present at a concentration between about 0.001 to about 1.0% (w/w). Sterile, single unit dose formulation of the present invention would not require a preservative.

The inventive compositions may optionally include one or more co-solvents and/or viscosity building agent. Suitable agents include but are not limited to a nonionic water-soluble polymer, an agent that lubricates, “wets”, and approximates the consistency of endogenous tears, and/or an agent that aids in natural tear build-up, or otherwise provide temporary relief of dry eye symptoms. Exemplary non-limiting compounds include, without limitation, monomeric polyols, such as glycerol, propylene glycol, and ethylene glycol; polymeric polyols, such as polyethylene glycol; cellulose compounds, such as hydroxypropylmethyl cellulose (HPMC), carboxymethylcellulose sodium, and hydroxylpropylcellulose (HPC); dextrans, such as dextran 70; water soluble proteins, such as gelatin; vinyl polymers, such as polyvinyl alcohol (PVA), polyvinylpyrrolidone (PVP), and povidone; and carbomers, such as carbomer 934P, carbomer 941, carbomer 940, and carbomer 974P.

A “phospholipid carrier” or “artificial tears” formulation recommended for the treatment of dry eyes may be included in the pyrethroid-containing inventive compositions. Such phospholipid carrier or artificial tears carriers are aqueous formulations which: (i) comprise one or more phospholipid or other compound that lubricates, “wets”, and/or approximates the consistency of endogenous tears, aids in natural tear build-up, and/or otherwise provides temporary relief of dry eye symptoms and conditions upon ocular administration; (ii) are substantially clinically safe to use; and (iii) provide the appropriate delivery vehicle for the topical administration of an effective amount of pyrethroid. Non-limiting examples of artificial tears formulations useful as artificial tears carriers include commercial products such as Moisture Eyes™ Lubricant Eye Drops/Artificial Tears, Moisture Eyes™ Liquid Gel lubricant eye drops, Moisture Eyes™ Preservative Free Lubricant Eye Drops/Artificial Tears, and Moisture Eyes™ Liquid Gel Preservative Free Lubricant Eye Drops/Artificial Tears (Bausch & Lomb Incorporated, Rochester, N.Y.). Non-limiting examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.), the contents of each of which are incorporated by reference herein. Other phospholipid or artificial tears carriers will be known to one skilled in the art.

Optionally, other compounds may also be added to the ophthalmic formulations of the present invention to increase the viscosity of the carrier. Non-limiting examples of suitable viscosity-enhancing agents include, but are not limited to: polysaccharides, such as hyaluronic acid and its salts; chondroitin sulfate and its salts; dextrans; various polymers of the cellulose family; vinyl polymers; and acrylic acid polymers.

The present inventive compositions may be prepared as topical preparations, such as a gel, a solution, a suspension, an emulsion or an ointment. They may also be imbedded or otherwise incorporated into an ocular implant device, including but not limited to a reservoir, matrix, contact lens or other solid device. The compositions are prepared in a manner to render them sterile for use as ophthalmic drug delivery systems. Methods of sterile preparation for various delivery systems are known to one skilled in the art.

By way of example, a sterile pharmaceutical composition of the invention that is a gel is made by first preparing a sterile polyacrylate gel and then incorporating an effective amount of a sterile pyrethroid (e.g. lambda-cyhalothrin or analog thereof) into said gel. Alternatively, the pyrethroid (e.g. lambda cyhalothrin or an analog thereof) may be suspended with a part of a solution, which may contain a sterile tonicity agent, used for the production of a polyacrylate gel, and this suspension may then be homogenously mixed in with a separately sterilized polyacrylate gel.

Sterilization may be accomplished using a variety of steps. For example, a polyacrylate suspension may be made and then autoclaved under sterile conditions. This acrylate suspension is mixed with a sterile-filtrated solution of preserving agent, isotonicity agent, and chelating agent. After careful and through mixing of the starting materials, the addition of sterile-filtrated caustic soda solution initiates gel formation, and the gel is further subjected to agitation until it is homogenous. A pyrethroid (e.g. lambda-cyhalothrin or an analog thereof) is sterilized by dissolving the pyrethroid in a suitable amount of solvent, for example ethyl acetate, subjecting the solution to sterile filtration, and precipitating the pyrethroid, for example, through the addition of sterile water with an antimicrobial agent under aseptic conditions. The microbially sterile pyrethroid may then be triturated or ground to a powder with about three to ten times the amount of the gel base. The remaining amount of gel may then be incorporated in the concentrate by thorough mixing. The finished gel preparation is then conventionally decanted or drawn off under sterile conditions.

In an alternative variation of the above method, the microbially sterile pyrethroid (e.g. lambda-cyhalothrin or analog thereof) can be, to a large extent, suspended in a part of the aqueous solution of the tonicity agent. The polyacrylate gel can be made in a conventional manner with the remaining amount of isotonic agent and separately the isotonic suspension of the pyrethroid can be homogenously mixed with the polyacrylate under sterile conditions.

Exemplary ophthalmic solutions are set forth below; however, the invention is not limited as such. The formulations are typical of ophthalmic formulations in terms of carrier and optional components. Other formulations and methods of preparing sterile ophthalmic drug delivery systems are known in the art.

Formulation 1

Ingredient Amount

Phase I

Carbopol® 934P NF (a high molecular weight acrylic acid-based polymer) 0.25 gm

Purified Water 99.75 gm

Phase II

Propylene Glycol 5.0 gm

Ethylenediamine tetraacetic acid (EDTA) 0.1 mg

Lambda-cyhalothrin “x” gm (where x is an amount that when diluted in the final composition produces an effective therapeutic composition). For purposes of this invention, an “effective therapeutic composition” is one that reduces IOP to within normal limits for a human subject, generally between about 10-20±mm Hg.

Mix five parts of phase II with twenty parts of phase I for more than 15 minutes and adjust pH to 6.2-6.4 using 1 N NaOH by titration.

Formulation 2

Ingredient Amount

Phase I

Carbopol® 934P NF (a high molecular weight acrylic acid-based polymer) 0.25 gm

Purified Water 99.75 gm

Phase II

Propylene Glycol 3.0 gm

Triacetin 7.0 gm

Lambda-cyhalothrin x gm (where x is an amount that when diluted in the final composition produces an effective therapeutic composition)

EDTA 0.1 mg

Mix five parts of phase II with twenty parts of phase I for more than 15 minutes and adjust pH to 6.2-6.4 using 1 N NaOH by titration.

Formulation 3

Ingredient Amount

Phase I

Carbopol® 934P NF (a high molecular weight acrylic acid-based polymer) 0.25 gm

Purified Water 99.75 gm

Phase II

Propylene Glycol 7.0 gm

Glycerin 3.0 mg

Lambda-cyhalothrin x gm (where “x” is an amount that when diluted in the final composition produces an effective therapeutic composition)

EDTA 0.1 mg

BAK 01 (benzalkonium chloride)—0.2 mg

Mix five parts of phase II with twenty parts of phase I for more than 15 minutes and adjust pH to 6.2-6.4 using 1 N NaOH by titration.

Formulation 4—Gel Carrier

One method of making a gel carrier useful in the present invention is described below. Other methods of making ophthalmic gels are known in the art. To produce 500 gm of polyacrylate gel, 1.220 gm of polyacrylic acid (packaged under the trademark “Carbopol® 980 NF”) is carefully suspended, with the aid of an ultrasonic apparatus, in about 700 ml water for injection and autoclaved for 20 minutes at 1210° C. and 2 bar pressure. In 700 ml of sterile, injection-grade water is then dissolved 0.050 gm of benzalkonium chloride (BAK), 20.000 gm sorbitol and 0.050 gm of sodium EDTA (X 2H2O), which is then subjected to sterile filtering (Sartorios®, Cellulose nitrate filter, order no. 11307-50ACN, 0.2 μm) into a sterile vessel. The sterile-filtered salt solution is then mixed, with strong agitation, into the autoclaved polyacrylate suspension. Sterile water in the amount of 1958.121 gm is then added, and the solution is subjected to further agitation for 5 to 10 minutes. Subsequently, strong sodium hydroxide in the amount of 0.465 gm is dissolved in exactly 40 gm of injection-grade water. This caustic soda is then introduced drop-wise under agitation over a sterile filter (Millex-GS, 0.22 μm, SLGS 025 BS der Fa. Millipore). The mixture is agitated until the formation of a completely homogenous gel.

A microbially sterile lambda-cyhalothrin is obtained commercially or prepared on site, one method of which is described above. The material is then slowly and carefully mixed with about 30 to 50 gm of the gel. The gel is subjected to sterile filtration of the solution and separation with water containing a bactericide, under sterile conditions. Loteprednol etabonate (ALREX®, LOTEMAX®) is dissolved in the given amount of gel to separate the composition. All method steps are carried out under aseptic conditions. The prepared gel is likewise drawn off in tubes under aseptic conditions.

By an alternative method, microbially sterile lambda-cyhalothrin is suspended in a sterile-filtrated isotonic solution of 700 ml water, 0.050 gm benzalkonium chloride, 20.000 gm sorbitol and 0.050 gm of disodium EDTA. This solution is then, as already described, incorporated, under strong agitation, into the autoclaved polyacrylate suspension.

The present invention further contemplates alternative forms of pyrethroid-based treatments, including but not limited to a solid matrix, reservoir, matrix, contact lens, or other device for insertion or implantation into the eye. Alternatively, the pyrethroid may be in a form that is encapsulated, for example in a liposome, and then incorporated into a solution or suspension for administration. The invention is not limited by the particular formulations, delivery systems, preparations or methods set forth herein.

In preparing formulations according to the present invention, it is likely that formulations will need to be adjusted to meet the demands of producing commercial scale (larger) amounts of solutions, suspensions, emulsions, ointments or gels, which is well within the expertise of one skilled in the art.

According to the above methods and formulations, a pyrethroid compound may be administered or placed into the eye by any method known in the art, including, but not limited to, topical instillation, injection, or the insertion of an ocular implant device, including but not limited to a reservoir, matrix, contact lens or other implantable device, to provide sustained release of the drug into the eye over time. A pharmaceutical composition of the invention may be administered to an affected eye topically, for example, as eye drops (solution, suspension or emulsion) or as an ointment or gel.

Administration may be at least once a day, at least twice a day, at least once a week, at least twice a week, at least once a month, at least twice a month, at least six times a year, at least four times a year, at least twice a year, or at least once a year, and/or up to twice a day, up to three times a day, up to once a week, up to twice a week, up to three times a month, up to six times a year, or up to four times a year, depending on the particular dosage form or delivery system utilized. For example, if the pharmaceutical composition is administered as eye drops, 1-2 drops of a pharmaceutical composition comprising between about 0.001 to about 5.0% (w/w) or between about 0.001 to about 1.0% (w/w) or between about 0.01 to about 1.0% (w/w) of pyrethroid may be administered to the affected eye at a time. If the composition is administered through an ocular device, designed to release medication over time, less frequent administration would be required.

A treatment regimen using a pyrethroid composition according to the invention may be combined with other treatments for glaucoma, such as those discussed herein.

### EXAMPLES

Materials: The following materials were used in the examples:

Lambda-cyhalothrin (LC)

Dimethyl sulfoxide (DMSO)

Propylene glycol (PG)

Balanced Salt Solution (BSS)

Preparations: The desired composition was DMSO 4.0%, PG 3.0% and LC 10 mg/ml (1% (by weight)), based upon the total composition. The preparation of 2.5 ml of the desired composition is shown in Table 1.

A vehicle was prepared for use as a subsequent dilution (serial dilutions of the initial LC strength), by mixing 4.65 ml of BSS, 0.2 ml of DMSO, and 0.15 ml of PG, to yield a diluent vehicle comprising 4.0% DMSO and 3.0% PG, based upon the total composition. A total of 5 ml was prepared.

The initial base LC formulation according to the invention is set forth in Table 1 below. A maximum concentration of 1.0% (by weight) LC was prepared.

TABLE 1

| COMPONENT/AMOUNT | PREPARATION STEP |
|---|---|
| LC 25 mg. | Added to DMSO with mixing. |
| DMSO 0.1 ml | |
| PG 0.075 ml | Mixed with LC/DMSO Mixture |
| BSS 2.325 ml | Slowly added to 0.175 ml LC/ |
| | DMSO/PG mixture |
| Total 2.5 ml | Final Concentration 1.0% LC, |
| | 4.0% DMSO, 3.0% PG |

Methodology: Inventive compositions were prepared by simple mixing.

IOP was determined using an iCARE® rebound tonometer. Other commercially available IOP measuring devices (tonometers) may also be used.

Progressive dilutions of the composition set forth in Table 1 are shown in Table 2, below, starting with the original 2.5 ml volume preparation of Table 1. These subsequent dilutions were used in the canine experiments set forth below.

TABLE 2

| Vol. | Diluent | | Vol. | |
|---|---|---|---|---|
| remaining | Vehicle | Total | removed | |
| of previous | to add | Volume | to dropper | |
| % (ml) | (ml) | (ml) | (ml) | % LC |
| — | — | 2.500 | 0.250 | 1.0% (wt.) |
| | | (starting | | |
| | | volume) | | |
| 2.250 | 0.250 | 2.500 | 0.278 | 0.9% |
| 2.220 | 0.278 | 2.500 | 0.313 | 0.8% |
| 2.188 | 0.313 | 2.500 | 0.357 | 0.7% |
| 2.143 | 0.357 | 2.500 | 0.417 | 0.6% |
| 2.083 | 0.417 | 2.500 | 0.500 | 0.5% |
| 2.000 | 0.500 | 2.500 | 0.625 | 0.4% |
| 1.875 | 0.625 | 2.500 | 0.833 | 0.3% |
| 1.667 | 0.833 | 2.500 | 2.500 | 0.01% |

### Example 1

The experiments were conducted on canine subjects. Canine subjects have IOP's that are the same (˜10-20 mm/Hg) as that of a human subject. Canines and humans share a common susceptibility gene, SRBD1, for glaucoma risk. Consequently, canines are excellent surrogates to assess the results of the use of the inventive compositions on humans. The results of the experiments are set forth in Table 3, below, reflecting the concentration range of LC used as 0.002% to 0.100% (by weight).

TABLE 3

| | LC (and | TIME | TIME | IOP | IOP | |
|---|---|---|---|---|---|---|
| DAY | CONC) | GTT | IOP | OD | OS | AE |
| Day 1 | NO LC | NONE | 13:00 | 16 | 16 | 0 |
| Day 1 | NO LC | NONE | 13:30 | 16 | 16 | 0 |
| Day 4 | 0.002% | 8:30 | 9:50 | 8 | 8 | 0 |
| Day 5 | 0.002% | 9:00 | 9:50 | 8 | 8 | 0 |
| Day 5 | 0.002% | 14:30 | 15:00 | 8 | 9 | 0 |
| Day 6 | NO LC | NONE | 8:00 | 19 | 18 | 0 |
| Day 7 | NO LC | NONE | 9:00 | 16 | 17 | 0 |
| Day 7 | 0.020% | 9:00 | 9:30 | 10 | 9 | 0 |
| Day 7 | NO LC | NONE | 15:00 | 15 | 16 | 0 |
| Day 7 | 0.020% | 15:00 | 16:00 | 10 | 7 | 0 |
| Day 9 | NO LC | NONE | 9:00 | 16 | 19 | 0 |
| Day 9 | 0.050% | 9:00 | 9:30 | 11 | 11 | 0 |
| Day 9 | NO LC | NONE | 15:00 | 10 | 9 | 0 |
| Day 9 | 0.050% | 15:00 | 16:00 | 7 | 8 | 0 |
| Day 11 | NO LC | NONE | 8:45 | 12 | 11 | 0 |
| Day 11 | 0.100% | 9:00 | 9:42 | 9 | 10 | 0 |
| Day 11 | NO LC | NONE | 14:30 | 7 | 8 | 0 |
| Day 11 | 0.100% | 15:00 | 15:22 | 11 | 5 | 0 |
| Day 13 | NO LC | NONE | 12:15 | 11 | 15 | 0 |

Day = day of experiment
LC = lambda-cyhalothrin (concentration)
Time GTT = time of drop instillation
Time IOP is time that intraocular pressure was measured (based on 24 hour clock or military time)
IOP OD = intraocular pressure right eye
IOP OS = intraocular pressure left eye
AE = adverse event
0 = none.

The data show that there was a significant reduction in intraocular pressure after topical administration of LC that was dose related. There were no local or systemic adverse events (as measured by daily, complete ophthalmic examinations and toxicology studies of blood).

The data provide sufficient evidence using the canine model, with its inherent similar physiology to humans that the same effect would occur with topical LC in a human. The canine eye, specifically anatomy of the anterior segment, the maintenance of normal eye pressure and production of intraocular fluid is the same as in humans, making the canine an excellent model for translational research and to apply to the use of medications to humans.

### Example 2

Additional efficacy and safety experiments were conducted using an LC concentration of 0.600% (by weight) in the same animal model. The results are shown in Table 4, below. This additional data confirms in two animals the safety and efficacy of topical LC in reducing intraocular pressure.

TABLE 4

| | LC | Time | Time | IOP | IOP | |
|---|---|---|---|---|---|---|
| DAY | Conc. | GTT | IOP | OD | OS | AE |
| Day 1 | NO LC | NONE | 8:45 | 11 | 11 | 0 |
| Day 1 | 0.600% | 9:30 | 9:55 | 8 | 7 | 0 |
| Day 1 | NO LC | NONE | 11:30 | 6 | 5 | 0 |
| Day 1 | 0.600% | 12:00 | 12:30 | 8 | 8 | 0 |
| Day 1 | NO LC | NONE | 15:20 | 7 | 6 | 0 |
| Day 3 | NO LC | NONE | 9:10 | 10 | 9 | 0 |
| Day 3 | 0.600% | 9:45 | 10:15 | 6 | 7 | 0 |
| Day 3 | NO LC | NONE | 11:45 | 6 | 8 | 0 |
| Day 3 | 0.600% | 12:10 | 12:40 | 7 | 7 | 0 |
| Day 3 | NO LC | NONE | 15:20 | 7 | 8 | 0 |

### Example 3

A series of experiments was run in the same canine model, using LC concentrations, prepared as above, and ranging from 0.200% to 0.700% (by weight). The results over time are shown in Table 5, below.

TABLE 5

| | LC | Time | Time | IOP | IOP | |
|---|---|---|---|---|---|---|
| DAY | Conc. | GTT | IOD | OD | OS | AE |
| Day 1 | NO LC | NONE | 17:05 | 10 | 9 | 0 |
| Day 2 | NO LC | NONE | 7:40 | 11 | 12 | 0 |
| Day 2 | 0.200% | 7:40 | 8:45 | 8 | 7 | 0 |
| Day 2 | NO LC | NONE | 12:20 | 7 | 6 | 0 |
| Day 2 | 0.200% | 12:22 | | | | 0 |
| Day 2 | NO LC | NONE | 13:43 | 8 | 6 | 0 |
| Day 2 | NO LC | NONE | 15:50 | 7 | 6 | 0 |
| Day 2 | NO LC | NONE | 17:00 | 7 | 7 | 0 |
| Day 2 | 0.200% | 17:05 | | | | 0 |
| Day 3 | 0.300% | 7:25 | 7:25 | 13 | 10 | 0 |
| Day 3 | NO LC | NONE | 9:47 | 8 | 8 | 0 |
| Day 3 | NO LC | NONE | 12:20 | 7 | 6 | 0 |
| Day 3 | 0.300% | 12:25 | 13:50 | 6 | 7 | 0 |
| Day 3 | 0.300% | 17:40 | 17:30 | 9 | 8 | 0 |
| Day 4 | 0.400% | 7:25 | 7:20 | 12 | 11 | 0 |
| Day 4 | NO LC | NONE | 9:40 | 9 | 9 | 0 |
| Day 4 | 0.400% | 12:40 | 12:35 | 7 | 7 | 0 |
| Day 4 | NO LC | NONE | 14:10 | 7 | 6 | 0 |
| Day 4 | 0.400% | 17:25 | 17:20 | 7 | 7 | 0 |
| Day 5 | 0.500% | 7:30 | 7:20 | 9 | 10 | 0 |
| Day 5 | NO LC | | 9:35 | 6 | 6 | 0 |
| Day 5 | 0.500% | 12:40 | 12:35 | 7 | 7 | 0 |
| Day 5 | NO LC | | 14:15 | 7 | 8 | 0 |
| Day 5 | NO LC | | 17:00 | 7 | 7 | 0 |
| Day 8 | 0.600% | 7:30 | 7:00 | 7 | 7 | 0 |
| Day 8 | NO LC | | 9:40 | 4 | 5 | 0 |
| Day 8 | 0.600% | 12:05 | 12:00 | 6 | 6 | 0 |
| Day 8 | NO LC | | 13:15 | 5 | 5 | 0 |
| Day 8 | 0.600% | 17:00 | 16:30 | 7 | 8 | 0 |
| Day 9 | 0.700% | 7:05 | 7:00 | 8 | 7 | 0 |
| Day 9 | NO LC | | 8:15 | 8 | 9 | 0 |
| Day 9 | 0.700% | 12:25 | 12:20 | 7 | 7 | 1 |
| Day 9 | NO LC | | 13:30 | 6 | 6 | 1 |
| Day 9 | 0.700% | 17:05 | 17:00 | 6 | 5 | 1 |
| Day 10 | NO LC | | 7:20 | 10 | 9 | 0 |
| Day 10 | NO LC | | 13:00 | 9 | 7 | 0 |
| Day 10 | NO LC | | 15:30 | 7 | 6 | 0 |
| Day 11 | NO LC | | 13:05 | 7 | 7 | 0 |
| Day 11 | NO LC | | 13:00 | 9 | 7 | 0 |
| Day 11 | NO LC | | 15:30 | 6 | 5 | 0 |
| Day 12 | NO LC | | 9:40 | 9 | 8 | 0 |
| Day 12 | NO LC | | 13:15 | 7 | 6 | 0 |
| Day 15 | NO LC | | 7:40 | 7 | 7 | 0 |
| Day 16 | NO LC | | 16:00 | 12 | 13 | 0 |
| Day 17 | NO LC | | 8:00 | 13 | 13 | 0 |

Overall, the results show efficacy in reducing IOP by 35-40% using dosages ranging from 0.002% to 0.700% LC (by weight). No adverse events were noted, except for minimal, reversible inferior conjunctival hyperemia at the 0.700% dose.

The profound reduction of intraocular pressure after administration of LCL in combination with its safety provide a new, more efficacious pharmacologic therapy in humans for the treatment of diseases of the eyes causing high intraocular pressure and, where the intraocular pressure needs to be reduced to restore or prevent vision loss.

While in accordance with the Patent Statutes, the best mode and preferred embodiments have been set forth, the scope of the invention is not limited thereto, but rather by the scope of the attached claims.

1. (canceled)

2. (canceled)

3. (canceled)

4. (canceled)

5. (canceled)

6. (canceled)

7. (canceled)

8. (canceled)

9. (canceled)

10. An ophthalmic pharmaceutical composition comprising a pyrethroid compound dispersed in a suitable delivery system, wherein the pyrethroid compound is present in an amount effective for treating eye disorders characterized by increased intra-ocular pressure in a human subject.

11. The composition of claim 10, wherein the eye disorder is glaucoma.

12. The composition of claim 10, wherein the delivery system is an ophthalmic solution, suspension, gel or emulsion formulated for topical administration into the eye.

13. The composition as in any of claims 10, 11, 12, 20, 21, or 22, wherein the pyrethroid compound is lambda-cyhalothrin.

14. (canceled)

15. (canceled)

16. (canceled)

17. (canceled)

18. (canceled)

19. (canceled)

20. The composition of claim 10, wherein the delivery system is an ocular device containing the composition for insertion into the eye.

21. The composition of claim 21, wherein the ocular device is a reservoir, implant or contact lens.

22. The composition of claim 10, wherein the delivery system is a solution, suspension, gel or emulsion formulated for injection into the eye.
